(19) United States
(12) Patent Application Publication
(10) Pub. No.: US 2016/0045579 A1
(43) Pub. Date: Feb. 18, 2016

(54) DIABETES TREATMENT

(71) Applicants: Maria KOULMANDA, (US); BETH ISRAEL DEACONESS MEDICAL CENTER, Boston, MA (US)

(72) Inventor: Maria Koulmanda, Brookline, MA (US)

(21) Appl. No.: 14/777,032

(22) PCT Filed: Mar. 17, 2014
(86) PCT No.: PCT/US14/30488

§ 371 Date: Sep. 15, 2015

Related U.S. Application Data
(60) Provisional application No. 61/799,692, filed on Mar. 15, 2013.

(51) Int. Cl. A61K 38/57; C12Q 1/54; A61K 38/28

(52) U.S. Cl. CPC A61K 38/57; A61K 38/28; C12Q 1/54

(57) ABSTRACT

Methods and compositions for treating a patient who has recently developed diabetes are provided. The methods can include administering a tolerance-restoring agent to the patient and simultaneously administering intensive insulin therapy.

### FIELD OF THE INVENTION

This invention relates to methods and compositions useful for the treatment of diabetes, and more particularly to methods for restoring tolerance.

### BACKGROUND

Diabetes is a lifelong chronic disease. Type I diabetes, in which patient fail to produce insulin, typically strikes in childhood or adolescence. Type I diabetes is an autoimmune disease that results from the progressive destruction of insulin-producing pancreatic β cells by CD4+ and CD8+ T cells. No cure is available and despite the availability of insulin therapy, patients with type I diabetes remain at risk for long-term complications. There is a continuing need for curative therapies for Type 1 diabetes.

### SUMMARY

The present invention is based, in part, on our discovery that meticulous monitoring and maintenance of glucose levels within normal limits during the administration of certain anti-diabetic regimens cured Type 1 diabetes (T1D) in an diabetic NOD mouse model. Our work to date indicates the importance of meticulous control of blood glucose levels while the tolerance restoring agent is administered. This approach allowed us to eventually withdraw insulin therapy altogether. Accordingly, the invention features methods and compositions for optimizing tolerance restoring therapies for treatment of diabetes. More specifically, the methods of the invention include the administration of intensive insulin therapy in conjunction with a tolerance-restoring agent to a patient who has recently developed diabetes.

The methods include administering intensive insulin therapy along with a tolerance restoring agent. We may use the “intensive insulin therapy,” “meticulous control of blood glucose levels,” “tight glycemic control,” “tight glucose control,” and “tight diabetes control” interchangeably. These refer to a treatment regimen designed to keep blood glucose levels consistently within normal limits e.g., 50-120 mg/dL. Intensive insulin therapy requires close monitoring of blood sugar levels and frequent doses of insulin. Blood glucose levels may be assayed multiple times per day, for example 2, 3, 4, 5, 6, 7, or 8 times per day. Blood glucose levels are typically checked before and after eating and before bedtime. Insulin is administered whenever blood glucose levels fall outside the normal limits. The dose of insulin can be based on the observed blood glucose level, so that small deviations outside the normal range are treated with a relatively low dose of insulin, while larger deviations outside the normal range are treated with a relatively higher dose of insulin. The method of administration of insulin can vary and depends in part on the degree to which the patient's glucose levels fall outside the normal range. In some cases, an injection of short-acting insulin is applied, for example before a meal, and in other cases an injection of intermediate or long-acting insulin is needed, for example, before bedtime. Alternatively, an insulin pump can be used to deliver a continuous infusion of short-acting insulin and a bolus of insulin just before meals in order to cover the rise in blood sugar that typically occurs after eating.

Tolerance restoring therapies can vary, but these generally target a variety of mechanisms involved in the development of autoimmune disease and the loss of tolerance in T1D. Typically, it is thought that as few as 15-20% of β cells remain at the time of the first clinical symptoms of T1D. Left unchecked, this residual islet cell function/mass is generally short-lived due to continued immune-mediated β cell death. However, the preservation of even this reduced β cell mass has clear therapeutic benefits by enabling better control of blood glucose, reducing exogenous insulin requirements and thus reducing the risk of diabetes-related complications. Useful tolerance restoring agents can potentially induce tolerogenic effects that outlast generalized suppression of the immune system.

Exemplary tolerance restoring agents include α1 antitrypsin; combinations of agents that include a first agent that targets an interleukin-15 receptor (IL-15R), a second agent that targets an interleukin-2 receptor (IL-2R), and rapamycin; a tim-1, tim-2 or tim-4 modulator; and a TNF-α modulator. Combinations of agents that target an interleukin-15 receptor (IL-15R), a second agent that targets an interleukin-2 receptor (IL-2R), and rapamycin are described in U.S. Pat. No. 7,579,439, which is herein incorporated by reference.

The compositions disclosed herein are generally and variously useful for treatment of diabetes. A patient is effectively treated whenever a clinically beneficial result ensues. This may mean, for example, a complete resolution of the symptoms of a disease, a decrease in the severity of the symptoms of the disease, or a slowing of the disease's progression. These methods can include the steps of a) identifying a patient (e.g., a human patient) who has diabetes; and b) providing to the subject a therapeutically effective amount of a pharmaceutical composition of a tolerance restoring agent while simultaneously administering intensive insulin therapy. The present methods may also include a monitoring step to help optimize dosing and scheduling as well as predict outcome. We may use the terms “subject,” “individual” and “patient” interchangeably. While the present methods are certainly intended for application to human patients, the invention is not so limited. Domesticated animals, including, for example cats, dogs, horses, cows and other domesticated animals can also be treated.

While we believe we understand certain events that occur upon administration of compositions a tolerance restoring agent while simultaneously administering intensive insulin therapy, the methods of the present invention are not limited to those that work by affecting any particular cellular mechanism. Our working hypothesis is that strictly maintained euglycemia promotes tolerance by favorably altering the ratio or molecular phenotype of islet-destructive and islet-protective T cells. Intense insulin therapy may also prevent exhaustion and apoptosis of residual islets, thus preventing augmentation of tissue-destructive T cell response. Moreover, a tight blood glucose control is a key factor determining long-term outcomes and severity of end organ injury (renal, cardiovascular, neurological) in patients with diabetes.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is graph depicting the results of an experiment in which newly diabetic NOD mice received intensive insulin therapy along with AAT.

FIG. 2 is graph depicting the results of an experiment in which newly diabetic NOD mice were treated with insulin implants along with AAT.

### DETAILED DESCRIPTION

Provided herein is a method of treating a patient who has diabetes, the method comprising: a) administering a tolerance-restoring agent to the patient; and b) simultaneously administering intensive insulin therapy. The method can include the step of administering intensive insulin therapy before administering the tolerance-restoring agent.

The method can also include the step of identifying a patient who has diabetes. In some embodiments the patient can be newly identified as having diabetes.

Tolerance-Restoring Agents

A tolerance restoring agent can be any agent that mitigates the autoimmune attack on insulin-producing cells that is characteristic of T1D. The term “agent” is meant to encompass essentially any type of molecule that can be used as a therapeutic agent. Proteins, such as antibodies, fusion proteins, and soluble ligands, any of which may either be identical to a wild-type protein or contain a mutation (i.e., a deletion, addition, or substitution of one or more amino acid residues), and the nucleic acid molecules that encode them (or that are “antisense” to them; e.g., an oligonucleotide that is antisense to the nucleic acids that encode a target polypeptide, or a component (e.g., a subunit) of their receptors), are all “agents.” The agents of the invention can either be administered systemically, locally, or by way of cell-based therapies (i.e., an agent of the invention can be administered to a patient by administering a cell that expresses that agent to the patient). The cell can be a cell administered to the patient solely for the purpose of expressing the therapeutic agent. The cell can also be a cell of a cellular, tissue, or organ transplant. For example, transplanted cells (e.g., islet cells) or cells within tissues or organs (e.g., cells within a patch of skin or a liver, kidney, or heart) can be treated with an agent or transduced with a nucleic acid molecule that encodes an agent ex vivo (e.g., prior to transplantation). In this way, the transplanted cell produces its own immunosuppressive agents. For example, a cell with a desirable phenotype (e.g., an insulin producing cell) can be modified to include a gene producing one or more of the immunosuppresive factors of the invention. The transplanted cell, tissue, or organ can be treated either prior to or subsequent to transplantation. Methods of administering agents to patients (or to cells or organs in culture) are known and routinely used by those of ordinary skill in the art and are discussed further below.

A tolerance restoring agent can be α1-antitrypsin (AAT; sometimes abbreviated A1AT), which is also referred to as α1-proteinase inhibitor. AAT is a major serum serine-protease inhibitor that inhibits the enzymatic activity of numerous serine proteases including neutrophil elastase, cathespin G, proteinase 3, thrombin, trypsin and chymotrypsin. For example, one can administer an AAT polypeptide (e.g., a purified or recombinant AAT, such as human AAT) or a homolog, biologically active fragment, or other active mutant thereof α1 proteinase inhibitors are commercially available for the treatment of AAT deficiencies, and include ARALAST™, PROLASTIN™ and ZEMAIRA™. The AAT polypeptide or the biologically active fragment or mutant thereof can be of human origin and can be purified from human tissue or plasma. Alternatively, it can be recombinantly produced. For ease of reading, we do not repeat the phrase “or a biologically active fragment or mutant thereof” after each reference to AAT. It is to be understood that, whenever a full-length, naturally occurring AAT can be used, a biologically active fragment or other biologically active mutant thereof (e.g., a mutant in which one or more amino acid residues have be substituted) can also be used. Similarly, we do not repeat on each occasion that a naturally occurring polypeptide (e.g., AAT) can be purified from a natural source or recombinantly produced. It is to be understood that both forms may be useful. Similarly, we do not repeatedly specify that the polypeptide can be of human or non-human origin. While there may be advantages to administering a human protein, the invention is not so limited.

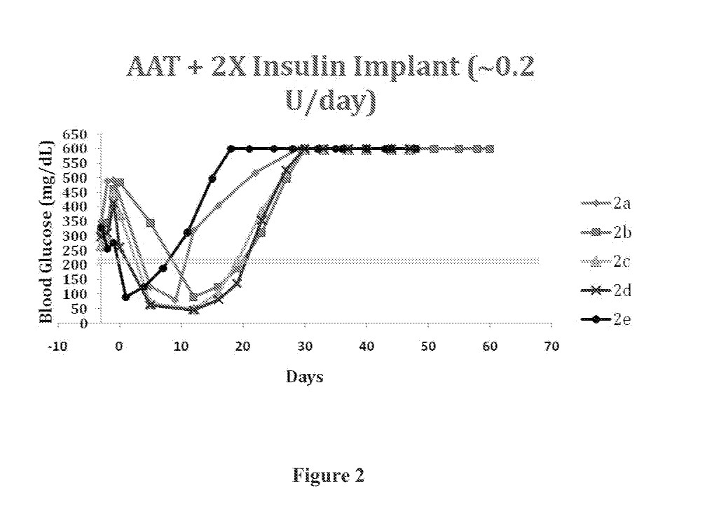

A tolerance-restoring agent can be an agent that targets IL-15 or an IL-15 receptor (IL-15R). A single agent can have multiple functional domains. Agents that target IL-15 or an IL-15R include agents that bind to (or otherwise interact with) IL-15, an IL-15R,or the nucleic acids that encode them as well as agents that bind to and subsequently destroy IL-15R-bearing cells, such as activated T cells. Thus, agents useful in achieving immune suppression can contain two functional moieties: a targeting moiety that targets the agent to an IL-15R-bearing cell and a target-cell depleting (e.g., lytic) moiety that leads to the elimination of the IL-15R-bearing cell. In one embodiment, the targeting moiety binds an IL-15R without effectively transducing a signal through that receptor. For example, the targeting moiety can include a mutant IL-15 polypeptide, and the target-cell depleting moiety can include the Fc region of an immunoglobulin molecule. The Fc region can be derived from an IgG, such as human IgG1, IgG2, IgG3, IgG4, or analogous mammalian IgGs or from an IgM, such as human IgM or analogous mammalian IgMs. In a preferred embodiment, the Fc region includes the hinge, CH2 and CH3 domains of human IgG1 or murine IgG2a. Although the invention is not limited to agents that work by any particular mechanism, it is believed that the Fc region mediates complement and phagocyte-driven elimination of IL-15R-bearing cells. Such agents can also include mutant IL-15 polypeptides that bind the IL-15 receptor complex with an affinity similar to wild-type IL-15, but fail to fully activate signal transduction. These mutant polypeptides compete effectively with wild-type IL-15 polypeptides and can inhibit one or more of the events that normally occur in response to IL-15 signaling, such as cellular proliferation. The “wild-type IL-15 polypeptide” referred to herein is a polypeptide that is identical to a naturally occurring IL-15 (e.g., a wild-type IL-15 polypeptide is shown in FIG. 2). In contrast, a “mutant IL-15 polypeptide” is a polypeptide that has at least one mutation relative to wild-type IL-15 and that inhibits at least one of the in vivo or in vitro activities that are usually promoted by wild-type IL-15.

A mutant IL-15 polypeptide that can be used according to the present invention will generally block at least 40%, more preferably at least 70%, and most preferably at least 90% of one or more of the activities of the wild-type IL-15 molecule. The ability of a mutant IL-15 polypeptide to block wild-type IL-15 activity can be assessed by functional assays. Further, mutant polypeptides of the invention can be defined according the particular percent identity they exhibit with wild-type IL-15. When examining the percent identity between two polypeptides, the length of the sequences compared will generally be at least 16 amino acids, preferably at least 20 amino acids, more preferably at least 25 amino acids, and most preferably at least 35 amino acids. The term “identity,” as used in reference to polypeptide or DNA sequences, refers to the identity between subunits (amino acid residues of proteins or nucleotides of DNA molecules) within the two polypeptide or DNA sequences being compared. When a subunit position in both of the molecules is occupied by the same monomeric subunit (i.e., the same amino acid residue or nucleotide), then the molecules are identical at that position. The similarity between two amino acid sequences or two nucleotide sequences is a direct function of the number of identical positions. Thus, a polypeptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Of course, many other polypeptides will meet the same criteria.

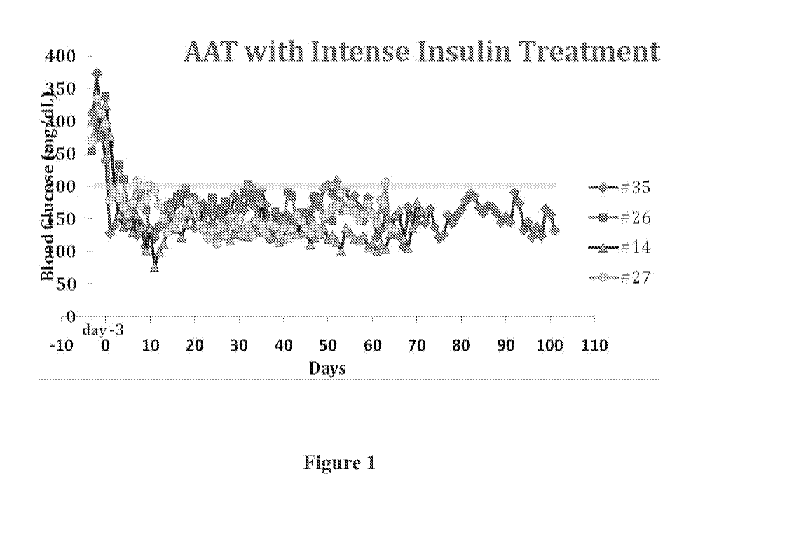

A mutant IL-15 polypeptide of the invention can be at least 65%, preferably at least 80%, more preferably at least 90%, and most preferably at least 95% (e.g., 96%, 97%, 98% or 99%) identical to wild-type IL-15. The mutation can consist of a change in the number or content of amino acid residues. For example, the mutant IL-15 can have a greater or a lesser number of amino acid residues than wild-type IL-15. Alternatively, or in addition, the mutant polypeptide can contain a substitution of one or more amino acid residues that are present in the wild-type IL-15. The mutant IL-15 polypeptide can differ from wild-type IL-15 by the addition, deletion, or substitution of a single amino acid residue, for example, an addition, deletion or substitution of the residue at position 156. Similarly, the mutant polypeptide can differ from wild-type by an addition, deletion, or substitution of two amino acid residues, for example, the residues at positions 156 and 149. For example, the mutant IL-15 polypeptide can differ from wild-type IL-15 by the substitution of aspartate for glutamine at residues 156 and 149 (as shown in FIG. 1). Mutant polypeptides useful as targeting agents, like wild-type IL-15, recognize and bind a component of the IL-15R. In one embodiment, the mutation of IL-15 is in the carboxy-terminal domain of the cytokine, which is believed to bind IL-2Rγ. (the IL-2 receptor subunit). Alternatively, or in addition, mutant IL-15 polypeptides can include one or more mutations within IL-2Rβ (the IL-2 receptor β subunit) binding domain.

In the event a mutant IL-15 polypeptide contains a substitution of one amino acid residue for another, the substitution can be, but is not necessarily, a conservative substitution, which includes a substitution within the following groups: glycine, alanine; valine, isoleucine, leucine; aspartic acid, glutamic acid; asparagine, glutamine; serine, threonine; lysine, arginine; and phenylalanine, tyrosine.

Instead of using, or in addition to using, an IL-15 targeting polypeptide (e.g., a mutant IL-15 polypeptide), the therapeutic agent can be an antibody. For example, IL-15 can be targeted (i.e., specifically bound) with an antibody. Similarly, the IL-15R can be targeted with antibodies that bind a component of the IL-15R (e.g., the IL-15Rα subunit). The methods by which antibodies, including humanized antibodies, can be generated against a component of the IL-15R are well known in the art. The antibodies preferably should be able to activate complement and phagocytosis, for example, human IgG3 and IgG1 (preferably the latter) subclasses, or murine IgG2a subclass.

The methods of the invention can also be carried out with compositions that contain: (a) two or more agents, each of which promote T cell death or (b) at least one agent that promotes T cell death and at least one agent that inhibits T cell proliferation. The agent that promotes T cell death can do so by promoting passive cell death, which occurs when a T cell is deprived of a factor required for its survival. IL-15 is one such agent (others are described below). Thus, agents that interfere with the ability of IL-15 to serve as a survival factor (e.g., an antibody that specifically binds to IL-15 or the IL-15 receptor) can be included in the compositions of the invention (e.g., a composition can include an agent that promotes AICD, an agent that promotes passive cell death (e.g., an anti-IL-15 antibody), and, optionally, an agent that inhibits T cell proliferation.

As described above, agents useful in achieving immune suppression can contain two functional moieties: a targeting moiety that targets the agent to an IL-15R-bearing cell (such as the mutant IL-15 molecule just described) and a target-cell depleting moiety that, for example, lyses or otherwise leads to the elimination of, the IL-15R-bearing cell. Thus, the agent can be a chimeric polypeptide that includes a mutant IL-15 polypeptide and a heterologous polypeptide such as the Fc region of the IgG and IgM subclasses of antibodies. The Fc region may include a mutation that inhibits complement fixation and Fc receptor binding, or it may be target-cell depleting (i.e., able to destroy cells by binding complement or by another mechanism, such as antibody-dependent complement lysis).

Reference is made herein to agents that “target” an interleukin or an interleukin receptor. Targeting occurs when an agent directly or indirectly binds to, or otherwise interacts with, an interleukin or an interleukin receptor in a way that affects the activity of the interleukin or the interleukin receptor. Activity can be assessed by those of ordinary skill in the art and with routine laboratory methods. For example, one can assess the strength of signal transduction or another downstream biological event that occurs, or would normally occur, following receptor binding. The activity generated by an agent that targets an interleukin or an interleukin receptor can be, but is not necessarily, different from the activity generated when a naturally occurring interleukin binds a naturally occurring interleukin receptor. For example, an agent that targets an IL-2 receptor falls within the scope of the invention even if that agent generates substantially the same activity that would occur had the receptor been bound by naturally occurring IL-2. When an agent generates activity that is substantially the same as, or greater than, the activity generated by a naturally occurring ligand, the agent can be described as a receptor agonist (the agent and the natural ligand being examined under the same conditions). When an agent generates activity that is less than the activity generated by a naturally occurring ligand, the agent can be described as an antagonist of the receptor (if the agent's primary interaction is with the receptor; e.g., mIL-15) or of the interleukin (if the agent's primary interaction is with the interleukin; e.g., an anti-IL-15 antibody). Here again, levels of activity are assessed by testing the agent and the naturally occurring receptor (or ligand) under the same conditions.

In addition, soluble Il-15Rα chain can be used as antagonist. While the IL-15 receptor complex consists of α, β, and subunits, the α chain alone displays a high affinity for IL-15. Thus, soluble IL-15R α chain will bind IL-15 and prevent IL-15 from binding to a cell surface-bound IL-15R complex. Thus, a soluble IL-15R α chain can act as a receptor-specific antagonist.

A tolerance-restoring agent can be an agent that targets an IL-2 or an IL-2 Receptor (IL-2R). To inhibit an immune response, the agents that target IL-15R-bearing cells, described above, can be administered with an agent that targets IL-2 or an IL-2R. An agent that is administered “with” another may be, but is not necessarily, administered at the same time or in the same manner (while this comment is stated in the context of a discussion of IL-2-related agents, it is applicable for any of the agents or molecules combined in the compositions of the invention). For example, an agent that targets an IL-15R may be administered before or after an agent that targets an IL-2R. Similarly, an agent that targets IL-15 or an IL15R can be administered ex vivo (to treat, for example, a cell, tissue, or organ that is slated for transplantation) while an agent that targets IL-2 or an IL-2R can be administered systemically (e.g., intravenously) to a patient (e.g. a patient who has received a transplant that was treated ex vivo with an agent that targets IL-15). Similarly, one can administer an agent that promotes AICD at a different time or in a different manner than an agent that inhibits cellular proliferation. Thus, in the methods of the invention, any of the agents or types of molecules that are combined in the compositions of the invention can be administered separately.

To inhibit an IL-2R, one can administer any agent that binds to and antagonizes IL-2 or an IL-2R. Agents that target IL-2 or an IL-2R include agents that bind to IL-2 or an IL-2R as well as agents that bind to and subsequently destroy IL-2R-bearing cells, such as activated T cells. As described above in the context of IL-15 targeting, agents useful in achieving immune suppression can contain a moiety that targets the agent to an IL-2R-bearing cell and a target-cell depleting (e.g., lytic) moiety that leads to the elimination of the IL-2R-bearing cell. For example, the targeting moiety can bind an IL-2R without effectively transducing a signal through that receptor.

Targeting agents, such as an IL-2/Fc agent (e.g., see Zheng et al., J. Immunol. 163:4041-4048, 1999) can be administered with an agent that prevents IL-2-mediated IL-2R signaling, such as rapamycin. Agents that inhibit cellular proliferation are well known to those of ordinary skill in the art. Alternatively or in addition to using an IL-2R targeting polypeptide (e.g., an IL-2 polypeptide), the therapeutic agent used in combination with an IL-15 antagonist can be an anti-IL-2 or an anti-IL-2R antibody (e.g., a humanized antibody) that antagonizes IL-2 or the IL-2R, respectively.

The methods of the invention can include compositions comprising: (a) two or more agents, each of which promote T cell death or (b) at least one agent that promotes T cell death and at least one agent that inhibits T cell proliferation. The agent that promotes T cell death can do so by promoting AICD (activation induced cell death), and such agents include IL-2 and molecules that function as IL-2 agonists. For example, IL-2/Fc, mutants of IL-2 that retain the ability to bind and transduce a signal through the IL-2 receptor, and antibodies that specifically bind and agonize the IL-2 receptor (e.g., an antibody that specifically binds the at subunit of the IL-2 receptor) can be included in the compositions of the invention. Other agents that promote AICD include Fas Ligand (FasL), which stimulates T cell death by activating the Fas signal transduction cascade on activated T cells, and biologically active mutants thereof.

A tolerance-restoring agent can be an agent that promotes passive T-cell death. Passive T cell death occurs when a T cell is deprived of an agent that is required for its survival. In addition to IL-15, factors including IL-4, IL-7, OX-40 ligand, IFN-β, 4-1BB and IGF-I are essential (i.e., T cells die in the absence of each of these factors. One can deprive T cells of one or more of these factors (IL-15, IL-4, IL-7, etc.) by, for example, exposing the cells, in vivo or in culture, to agents that selectively bind to one or more of the factors or otherwise prevent them from interacting with the T cell as they normally would (the result of the deprivation being passive cell death).

A tolerance-restoring agent can be an agent that that promotes antibody-dependent cell-mediated cytotoxicity (ADCC) or complement-dependent cytolysis (CDC). ADCC and CDC can be provoked by agents that bind to the T cell surface and that contain an Fc portion of an immunoglobulin molecule that activates ADCC or CDC. Examples of such agents include antibodies that bind to cell surface structures that are expressed on activated immune cells (e.g., cell surface receptors such as CD154, the IL-2 receptor, and the IL-15 receptor). In addition, one can use a ligand/Fc chimeric fusion protein, which binds to receptor proteins on the surface of activated cells (e.g., an IL-2/Fc or a mIL-15/Fc). Given these examples, other suitable agents will be apparent to those of ordinary skill in the art.

A tolerance-restoring agent can be an agent that inhibits cellular proliferation. Agents that inhibit cellular proliferation include rapamycin (Sirolimus), mycophenolate mofetil (MMF), azathioprine, and any other of the agents that are known to be useful for the treatment of hyperproliferative disorders (such as cancer). Well-characterized chemotherapeutics include agents that inhibit nucleic acid metabolism (such as purine and pyrimidine biosynthesis inhibitors, RNA synthesis inhibitors, and DNA binding, DNA modifying, or intercalating agents). These agents are especially useful when the composition used to, for example, inhibit an immune response, also contains an agent such as IL-2/Fc, which not only promotes AICD but also stimulates T cell proliferation.

Agents that inhibit cellular proliferation also include folic acid antimetabolites such as methotrexate (MTX) and pyrimethamine; purine antimetabolites (such as 6-mercaptopurine (6-MP) and azathioprine) and pyrimidine antagonists such as cytarabine (ara-C), 5-azacytidine, and 5-fluorouracil (these categories were mentioned above); alkylating and other DNA-linking agents (e.g., cyclophosphamide (CPA); mitomycin C, and Doxorubicin (Adriamycin)); vinca alkaloids (e.g., vincristine); and calcineurin inhibitors (e.g., Cyclosporin A, FK506, and Brequinar).

Other agents that can be used to inhibit cellular proliferation include agents that interfere directly with proteins involved in cell cycle regulation (such as anti-CDKs (Cell Division Kinase) or anti-cyclins) or proteins that affect cell proliferation check points (all proliferating cells have check points at different stages of the cell cycle that prevent them from entering the next stage of the cell division cycle (CDC) before they have concluded the previous step). Pathways that feed into check point controls include DNA-, RNA- and protein-synthesis inhibitors (e.g., S6 kinase and PI-3-kinase inhibitors). Cytokinesis inhibitors can also be used.

The compositions disclosed herein are generally and variously useful for treatment of patients who have recently developed diabetes. A patient is effectively treated whenever a clinically beneficial result ensues. This may mean, for example, a complete resolution of the symptoms of diabetes, a decrease in the severity of the symptoms of diabetes, or a slowing of diabetes' progression. These methods can further include the steps of a) identifying a subject (e.g., a patient and, more specifically, a human patient) who has diabetes; and b) providing to the subject a composition comprising a tolerance restoring agent, along with a regime of intensive insulin therapy. An amount of such a a tolerance restoring agent provided to the subject that results in a complete resolution of the symptoms of a disease, a decrease in the severity of the symptoms of the disease, or a slowing of the disease's progression is considered a therapeutically effective amount. The present methods may also include a monitoring step to help optimize dosing and scheduling as well as predict outcome.

Patients amenable to treatment include patients who have recently developed diabetes. The destruction of β-cells that is characteristic of diabetes generally occurs over a period of a few years. Patients who have recently developed diabetes retain some β-cell function, for example 5%, 10%, 20%, 25%, 30%, 35%, 40%, 45%, 50%, 55%, 60%, 65%, 70%, 75%, 80%, 85%, 90%, or 95% of the β-cell function found in individuals who do not have diabetes. Thus, the methods of the invention are useful for those patients who retain some β-cell function. Residual β-cell function can be assessed using any art know method, for example, measurement of insulin secretion, c-peptide levels or glucose tolerance. A patient who has recently developed diabetes can be a patient who is suspected as having diabetes or can be a patient who has recently been diagnosed with diabetes. In some embodiment a patient who has recently been diagnosed with diabetes is a pateint who is within a few days, e.g., 1, 2, 3, 4, 5, 6, 7, 8, 9, 10 or more days; a few weeks, e.g., 1, 2, 3, 4, 5, 6, 7, 8, 9, 10 or more weeks; a few months e.g., 1, 2, 3, 4, 5, 6, 7, 8, 9, 10 or more weeks; or a few years e.g., 1, 2, 3, 4, 5, 6, 7, 8, 9, 10 or more years of diagnosis.

The methods disclosed herein can be applied to a wide range of species, e.g., humans, non-human primates (e.g., monkeys), horses or other livestock, dogs, cats or other mammals kept as pets, rats, mice, or other laboratory animals. The compounds described herein are useful in therapeutic compositions and regimens or for the manufacture of a medicament for use in treatment of diseases or conditions as described herein (e.g., diabetes as disclosed herein). Such methods can include a tolerance-restoring agent for use in treating recently developed diabetes, wherein the tolerance-restoring agent is administered simultaneously with intensive insulin therapy. Alternatively or in addition, such methods can include intensive insulin therapy for use in treating recently developed diabetes, wherein the intensive insulin therapy is administered simultaneously with a tolerance-restoring agent.

The tolerance restoring agents described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Suitable dosages are in the range of 0.01-1,000 mg/kg. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The compositions may also be administered with another therapeutic agent, such as insulin. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The intensive insulin therapy can include a) measuring the glucose level in a biological sample from the patient, wherein an elevated glucose level relative to a predetermined range indicates the subject is hyperglycemic; b) optionally measuring the levels of one or more glucose-related markers; c) administering a blood glucose regulating agent in an amount sufficient to reduce blood glucose to a euglycemic level. The glucose level can be measured before and/or after the patient consumes a meal. In some embodiments, the predetermined range for glucose levels is from about 70 mg/dL to about 130 mg/dL. In some embodiments, the predetermined range is from about 60 mg/dL to about 180 mg/dL. Glucose related-markers are also useful for in intensive insulin therapy. In some embodiment, an elevated level of the glucose related marker, e.g, glycated hemoglobin, relative to a predetermined range indicates the patient is hyperglycemic. The predetermined range of glycated hemoglobin can be from about 4% to about 7%. Glucose levels can be measured in a biological sample, e.g., a blood, plasma, serum, or urine sample.

The tolerance-restoring agent can be administered until a symptom of diabetes improves. In some embodiments, the dosage of the blood glucose regulating agent can be progressively lowered as one or more symptoms of diabetes improves.

Also provided is a method of preserving or restoring β-islet cell function in a patient who has diabetes, the method comprising: a) administering a tolerance-restoring agent to the patient; and b) simultaneously administering intensive insulin therapy.

Also provided are kits comprising a measured amount of one or more of a tolerance restoring agent, a blood glucose regulating agent and one or more items selected from the group consisting of packaging material, a package insert comprising instructions for use, a sterile fluid, and a sterile container.

### EXAMPLES

### Example 1

### Materials and Methods

Female NOD (NOD/LtJx) mice and NOD.SCID (NOD.CB17-Prkdcscid/J) mice were purchased from Jackson Laboratories (Bar Harbor, Me.) at 4 wks of age and maintained under pathogen-free conditions at the Harvard Institutes of Medicine (Boston, Mass.). The Harvard Medical School institutional review board approved all animal studies.

Blood glucose levels of NOD mice were monitored twice weekly with the Accu-Check blood glucose monitor system (Roche, Indianapolis, Ind.). When non-fasting blood glucose levels were in excess of 200 mg/dl on three consecutive measurements a diagnosis of new onset of diabetes was made. Mice with blood glucose levels between 250 to 350 mg/dl were used for the experiment described below. Previous morphometric analysis of the insulin positive mass of the pancreatic islets revealed that NODs with blood glucose levels between 250 mg/dl-350 mg/dl have about 25% of the insulin positive beta cell mass of non-autoimmune NOD.SCID mice. The 25% residual beta cell mass is similar to that found in newly diagnosed patients with T1DM. Mice with blood glucose levels over 350 mg/dl just do not have meaningful number of surviving islets

### Example 2

### The Effect of Intense Insulin Treatment on α1 Anti-Trypsin Treatment

New onset diabetic NOD mice were divided into two groups and treated with α1anti-trypsin while simultaneously receiving intense insulin treatment (Group 1) or implantable insulin pellets (Group 2).

For the animals in Group 1, insulin doses were calibrated on the basis of three blood glucose measurements per day. The goal was to maintain blood glucose levels between 100-160 mg/dl. For blood glucose levels in excess of 160 mg/dl, NPH insulin was administered i.p. in doses ranging from 1 to 4 International Units depending upon the magnitude of hyperglycemia. For blood glucose level between 160 and 200 mg/dl, 1 IU was given. For blood glucose levels between 200 and 250 mg/dl, 2 IU of NPH insulin were given. For blood glucose levels between 250 and 300 mg/dl, 2 IU of NPH insulin were given. For blood glucose levels between 300 and 350 mg/dl, 3 IU of NPH insulin were given. For blood glucose levels in excess of 350 mg/dl, 4 IU of NPH insulin were given. Mice with high blood glucose levels in excess of 400 mg/dl for 2 days also received 0.2-0.4 ml warm normal saline subcutaneously in order to treat glycosuria-associated dehydration. We had found that mice with blood glucose levels over 400 mg/dl became dehydrated from the glycosuria and were prone to develop ketoacidosis and did not respond to insulin.

The animals in Group 2, LinBit™ sustained release insulin implants were used for management of hyperglycemia. LinBit™ sustained release insulin implants were purchased from LinShin Canada Inc. The LinBit™ implants (3-mm in length and 2 mm in diameter) were made from an admixture of insulin and micro-recrystallized palmitic acid and were erodible in vivo after being placed subcutaneously. The insulin release rate was about 0.1 U/day/implant for the time period over 2-4 weeks.

In our experiments, one or 2 LinBit™ implants were placed subcutaneously under the mid dorsal skin in newly onset diabetic mice with 3 previous measurements of blood glucose levels >200 mg/dL. The mice were anesthesized with ketamine/xylazine and the hair on the dorsal skin from a spot about 1 cm² was clipped using a small scissors with curved tips. After skin disinfection with 70% ETOH, small skin incision was made with surgical scissors. Implants were sterilized by short exposure to UV and then held by pinzette and inserted through the skin incision, after dissection of subcutaneous space with surgical scissors. One wound clip was used for closure.

Aralast NP, a human α1-proteinase inhibitor derived from pooled human plasma, was purchased from Baxter. Aralast NP was administered to animals in both Groups 1 and 2 at a dose of 2 mg intraperitoneally every 3 days for a total of five injections. The last day of treatment was day 15.

As shown in FIG. 1, blood glucose levels of the animals receiving intense insulin therapy along with α1 anti-trypsin (“AAT”) fell to normoglycemic levels shortly after treatment began and remained at normal levels for the entire duration of the experiment (60-100 days depending on the individual animal). In contrast, although blood glucose levels of the animals receiving the insulin implants along with α1 anti-trypsin fell to normoglycemic levels shortly after treatment began, the levels began to rise between day 15 and day 20 and remained between 500 mg/dl and 500 mg/dl for the entire duration of the experiment.

These data suggested that α1 anti-trypsin had a curative effect on diabetic NOD mice when glucose levels were meticulously monitored and maintained within normal limits (50-120 mg/dL) by intensive insulin therapy administered as early as the diagnosis of type I diabetes was made. This “bridging” insulin therapy was gradually withdrawn as the diabetic state was cured by tolerizing regimen. In contrast, if hyperglycemic state of the new onset diabetic NOD mice was managed by subcutaneously placed insulin pellets, the tolerance-restoring regimen was less effective. The insulin pellets appear to modify but not control hyperglycemia. These data also suggested that hyperglycemia may negates the therapeutic benefit of tolerance-restoring therapies in NOD mice.

A number of embodiments of the invention have been described. Nevertheless, it will be understood that various modifications may be made without departing from the spirit and scope of the invention. Accordingly, other embodiments are within the scope of the following claims.

1. A method of treating a patient who has recently developed diabetes, the method comprising:
a) administering a tolerance-restoring agent to the patient; and
b) simultaneously administering intensive insulin therapy.

2. The method of claim 1, wherein the step of administering intensive insulin therapy is carried out before administering the tolerance-restoring agent.

3. The method of claim 1, further comprising the step of identifying a patient who has diabetes.

4. The method of claim 1, wherein the patient retains residual β-cell function.

5. The method of claim 1, wherein the tolerance-restoring agent is selected from the group consisting of (a) a first agent that targets an interleukin-15 receptor (IL-15R), a second agent that targets an interleukin-2 receptor (IL-2R), and rapamycin; (b) alpha 1 antitrypsin; (c) a tim-1, tim-2 or tim-4 modulators; and (d) a TNF-alpha modulator.

6. The method of claim 1, wherein step (b) is carried out one or more times a day.

7. The method of claim 1, wherein the intensive insulin therapy comprises:
a) measuring the glucose level in a biological sample from the patient, wherein an elevated glucose level relative to a predetermined range indicates the subject is hyperglycemic;
b) optionally measuring the levels of one or more glucose-related markers; and
c) administering a blood glucose regulating agent in an amount sufficient to reduce blood glucose to a euglycemic level.

8. The method of claim 7, wherein the glucose level is measured before or after the patient consumes a meal.

9.-10. (canceled)

11. The method of claim 7, wherein the predetermined range is from about 60 mg/dL to about 180 mg/dL.

12. The method of claim 7, wherein an elevated level of the glucose-related marker relative to a predetermined range indicates the subject is hyperglycemic.

13. The method of claim 7, wherein the glucose-related marker is glycated hemoglobin.

14. The method of claim 13, wherein the predetermined range of glycated hemoglobin is from about 4% to about 7%.

15. (canceled)

16. The method of claim 15, wherein the biological sample is a blood, plasma, serum, or urine sample.

17. The method of claim 7, wherein the blood glucose regulating agent is insulin or an insulin analogue.

18. The method of claim 17, wherein the insulin is rapid-acting insulin, intermediate-acting insulin, or long-acting insulin.

19. The method of claim 1, wherein the tolerance-restoring agent is administered until a symptom of diabetes improves.

20. The method of claim 7, wherein the dosage of the blood glucose regulating agent is progressively lowered as one or more symptoms of diabetes improves.

21. The method of claim 1, wherein the diabetes is type I diabetes.

22. A method of preserving or restoring β-islet cell function in a patient who has diabetes, the method comprising:
a) administering a tolerance-restoring agent to the patient; and
b) simultaneously administering intensive insulin therapy.

23. (canceled)

24. A kit comprising a measured amount of one or more of a tolerance restoring agent, a blood glucose regulating agent and one or more items selected from the group consisting of packaging material, a package insert comprising instructions for use, a sterile fluid, and a sterile container.

25.-34. (canceled)
